(19) United States
(12) Patent Application Publication
(10) Pub. No.: US 2006/0047316 A1
(43) Pub. Date: Mar. 2, 2006

(54) System for the treatment of migraine headaches

(76) Inventors: Robert E. Fischell, Dayton, MD (US); Scott J.S. Fischell, Glenelg, MD (US); Adrian R.M. Upton, Dundas (CA)

Correspondence Address: Dr. Robert E. Fischell, 14600 Viburnum Drive, Dayton, MD, 21036, US

(21) Appl. No.: 10/929,586

(22) Filed: Aug. 31, 2004

(51) Int. Cl. A61N 1/39

(52) U.S. Cl. 607/1

(57) ABSTRACT

Disclosed is a system and method for treating migraine headaches employing a readily portable magnetic pulse system that has a head unit connected to a table unit by means of a connecting cable. When a patient senses either the aura of a migraine headache or an ongoing headache, he would turn the table unit on and then press a charge button to charge at least one capacitor in the head unit. When the at least one capacitor is fully charged, the patient would then place the head unit onto his head and then press a button switch that causes the at least one capacitor to discharge into an electromagnetic coil located in the head unit. The high intensity electric current in the electromagnetic coil would then create a high intensity, short duration magnetic pulse onto the patient's brain which can decrease the intensity or duration of the migraine headache.

### FIELD OF USE

This invention is in the field of medical devices for the treatment of migraine headaches.

### BACKGROUND OF THE INVENTION

Migraine headaches occur in approximately 12% of the world population. Therefore, in the United States in the year 2004 there are approximately 30 million people who suffer from this affliction. Although medicines have been created that significantly diminish the suffering of migraine patients, these medicines often have highly undesirable side effects and many patients do not obtain satisfactory relief from the severe headache pain, nausea and other discomforts associated with migraine. Furthermore, migraine headaches are typically treated after they have become painful, i.e., the treatment is often ineffective in preventing the onset of the migraine headache. Other than some drugs that are efficacious for some patients, there is currently no known treatment for migraine headaches that can be applied after a patient detects an aura of that headache to prevent the occurrence of pain and other undesirable manifestations of that migraine headache. A non-invasive, non-drug method for preventing the occurrence of migraine headaches would be a remarkable boon for those millions of people all over the world who suffer from these painful experiences.

In 1985, A. T. Barker, et al (Lancet, 1985, pp. 1105-1107) described the use of a coil placed over the scalp which produced a high intensity, time varying, magnetic field. This magnetic field produces an electric current in the cortex of the human brain which can in turn produce certain effects on brain neurons. This type of system has been given the name Transcranial Magnetic Stimulation (TMS). If repetitive magnetic pulses are applied in this manner, it has been given the name rTMS.

In the journal Neurology (Apr. 11, 2000, pp. 1529-1531) it has been reported by B. Boroojerdi, et al that rTMS at a rate of one pulse per second can create a reduction of the excitability of the neurons of the human visual cortex. However, that article did not indicate that TMS or rTMS can be used for the preventing the occurrence of migraine headaches or diminishing the intensity or duration of a migraine headache.

In U.S. Pat. No. 6,402,678, Robert E. Fischell et al describe means and methods for the treatment of migraine headaches using a portable device that is placed onto the patient's head. This device is used to create a magnetic pulse that acts upon the neurons of the brain and can eliminate both the aura of a migraine headache and a migraine headache after it has started. However, since the entire device is placed onto the patient's head, it is quite heavy and somewhat awkward for the patient's use. Furthermore, since the triggering controls are also located on the head mounted device, their operation is also somewhat awkward.

### SUMMARY OF THE INVENTION

This present invention teaches means and methods for the treatment of migraine headaches for those patients who experience a distinct aura before the actual occurrence of the symptoms of the migraine headache and also for those patients whose migraine headache has already started. It is estimated that approximately 40% of all migraine patients have a distinct aura that is a precursor of a migraine headache. Approximately half of those patients have a visual aura that typically begins as a small pattern of scintillating colored lights that have the appearance of wiggling worms. Over a time period of between 20 and 30 minutes, the pattern enlarges until it occupies nearly the entire visual field. During this time period, the patient might also completely lose all or part of his visual field. At the end of this visual aura, most migraine patients have a severe headache that is often accompanied by other symptoms such as nausea, vomiting and other unpleasant feelings. Many migraine patients who don't have a visual aura have some other precursor of a migraine that can be perceived by themselves or others from minutes to hours before the actual start of the headache. By treatment with a high intensity, short duration magnetic pulse (or pulses) when any precursor of a migraine headache occurs, some migraine headaches can be reduced as to either or both intensity and duration or completely eliminated.

The visual aura of a migraine headache is a result of the spatial progression of a band of brain cells that are excited in that band across one half of the brain's occipital lobe. This band moves in an anterior direction at the rate of approximately 2-5 mm per minute. It is this excited band of neurons of the brain that produces the scintillating colored lights that are perceived by the patient as a visual aura that is a precursor of a migraine headache. Behind this leading band of excited neurons, a spreading region of neurons with depressed electrical excitability occurs. This phenomenon is known as “the spreading depression of Leao”.

Recent tests with human subjects have shown that the advancement of the band of excited neurons can be eliminated before the aura has completed its 20 to 30 minute time duration period, and by that means, the migraine headache never occurs. This is accomplished by applying a high intensity, short duration magnetic pulse onto the neurons of the brain which causes mostly depolarization and some polarization of these neurons, which in turn results in elimination of the visual aura and prevention of the migraine headache.

Although the portable magnetic pulse system as described herein is excellent for the treatment of an aura that frequently precedes a migraine headache, the application of a high intensity, short duration magnetic pulse can also be used for the treatment of an ongoing migraine headache. Although some migraine headaches can be treated with a single magnetic pulse, it is envisioned that some auras and some ongoing migraine headaches can best be treated by the application of a multiplicity of magnetic pulses.

One aspect of the invention that is disclosed herein is a non-invasive, externally applied magnetic pulse system that consists of two major parts; a table unit that would typically be placed on a table and a head unit that is designed to apply the magnetic pulse to the neurons of the brain. For the treatment of aura, the head unit is designed to be placed on or near the patient's head in the region of the brain where the aura originates (e.g., the occipital lobe) as soon as possible after the patient becomes aware of a visual (or any other type) aura that is the precursor of their migraine headache. For patients whose aura originates from a region of the cerebral cortex that is not the occipital lobe, the magnetic pulse can be applied to that region of the brain. By the use of a high intensity, short duration magnetic pulse, a sufficiently high electrical current is created at the location of the advancing band of excited neurons so as to depolarize many of those neurons thereby terminating the aura before it is able to progress into a migraine headache. Mostly depolarization and some polarization of neurons can also be used to treat an ongoing migraine headache. This is because depolarized neurons become refractory after TMS is applied. This is analogous to cutting down or burning the trees in front of a forest fire in order to prevent the spread of that forest fire.

A single high intensity, short duration, TMS magnetic pulse can be used to reduce cerebral cortex excitability thereby breaking up the advancing band of excited neurons that is the cause of the visible aura of a migraine headache. Since an aura has a time duration that is typically at least 20 minutes, the patient has a sufficient time period for placing the head unit in the appropriate position for it to be effective in depolarizing the advancing band of excited brain neurons. If there is an ongoing headache, the patient can place the head unit onto his or her head as soon as possible. For either the treatment of any type of aura or for an ongoing headache, the magnetic pulse system can be used to apply a multiplicity of pulses if the first treatment with a single pulse does not eliminate or significantly reduce the headache.

It should also be noted that stimulation of the scalp or trigeminal nerve might also have an effect in preventing or decreasing the severity or time duration of a migraine headache for at least some patients. Scalp or trigeminal nerve stimulation may act as a conditioning response that becomes associated with the migraine process. Furthermore, it is believed that single or multiple TMS pulses can be used for the treatment of facial pain including trigeminal neuralgia. For this application, the high intensity, short duration magnetic pulse (or pulses) can be applied at the site of the pain, or onto the trigeminal nerve or onto the patient's brain.

Since the band of excited neurons that create a visual aura moves from the back of the head in an anterior direction, and since either the left or right half of the occipital lobe might be involved, the head unit would optimally be placed along the posterior-anterior centerline at the top of the head. If it is known that a particular patient has the spreading depression on either the right or the left half of the occipital lobe, then the head unit might be placed only on that region where the spreading depression occurs. If the aura originates from a part of the cerebral cortex that is not the occipital lobe, then the magnetic pulse can be appropriately placed to depolarize neurons in that location. It is expected that the patient can be trained to recognize the symptoms from a particular area of the brain so that the head unit can be placed in an optimum location to prevent the occurrence of a migraine if it is precede by an aura, or reduce its intensity and/or duration for an ongoing migraine headache.

A sufficiently intense, short duration magnetic pulse must be created to treat a migraine headache. The intensity of the magnetic field at the surface of the brain should be between 0.1 and 2.0 Tesla. The frequency rate of the magnetic pulses should be between one per second and one per minute. With some patients a single, short duration pulse may be all that is required to stop an advancing band of excited neurons from proceeding to a full-blown migraine headache or to reduce the intensity and/or duration of an ongoing migraine headache. For some patients, at least two high intensity, short duration magnetic pulses may be required. By applying a time varying magnetic field to the neurons of the cerebral cortex (and also the neurons in the scalp and/or the trigeminal nerve), a patient should be able to actually prevent the occurrence of a migraine headache or decrease its intensity and duration after the headache has started. As previously described, the optimum placement for head unit of the magnetic pulse system when there is a visual aura is over the patient's occipital lobe. For other auras, the optimum placement for the head unit will be that place on the patient's head that is in closest proximity to the portion of the patient's brain where that aura originates. For an ongoing headache, the patient can discover by trial and error where the optimum location for applying the magnetic pulse is located. One method that has been reasonably successful is to apply the magnetic pulse to that region of the patient's brain where he is feeling the most pain.

Although the patient is referred to herein by use of the masculine words “his,” “he” or “himself,” it should be understood that the patient can be either a man or a woman.

Thus, an objective of this invention is to prevent the occurrence of a migraine headache by creating a high intensity, short duration magnetic pulse by means of a head unit placed onto the scalp of a patient who has an aura which is a precursor of a migraine headache; the head unit being designed to cause depolarization of at least some of the neurons in the cerebral cortex where the aura originates.

Another object of this invention is to decrease the weight of the portion of the magnetic pulse system that has to be placed onto the head (or neck) by limiting the number of components of the system that are placed into the head unit and by placing as many of the additional components of the system that are required into table unit.

Still another objective of this invention is to reduce the severity and/or duration of an ongoing migraine headache by applying a high intensity, short duration magnetic pulse to a patient's brain or trigeminal nerve by means of a head unit that receives its power from a table unit.

Still another object of the invention is to have at least most if not all of the operating controls for the magnetic pulse system placed on the table unit where they are most easily operated by the patient.

Still another object of the invention is to have the magnetic pulse system obtain its electrical power from either a-c line power, from the cigarette lighter in an automobile or from an internal or external rechargeable or disposable battery.

Still another object of the invention is to place the capacitors needed for providing a high intensity electrical current pulse into the head unit of the magnetic pulse system so that the electrical connecting wire to the table unit can be lightweight and flexible.

Still another object of the invention is the placement of the capacitors needed for providing a high intensity electrical current pulse in the handle of the head unit of the magnetic pulse system so that the capacitors can be located in close proximity to the magnetic coil that requires the high current from the capacitors to generate the high intensity magnetic pulse.

Still another object of the system is to have the magnetic coil of the head unit shaped in the form of a spherical sector so as to maximize the intensity of the magnetic pulse onto the neurons of the brain.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1 is an illustration of the magnetic pulse system that includes a head unit and a table unit.

FIG. 2 is a top view of the table unit showing the various switches and indicator lights of the magnetic pulse system.

FIG. 3 is a top view of the head unit of the magnetic pulse system.

FIG. 4 is a partial longitudinal cross section of the head unit of the magnetic pulse system.

FIG. 5 is a block diagram of the magnetic pulse system.

FIG. 6 is a graph of the shape of the high intensity, short duration magnetic pulse.

### DETAILED DESCRIPTION OF THE INVENTION

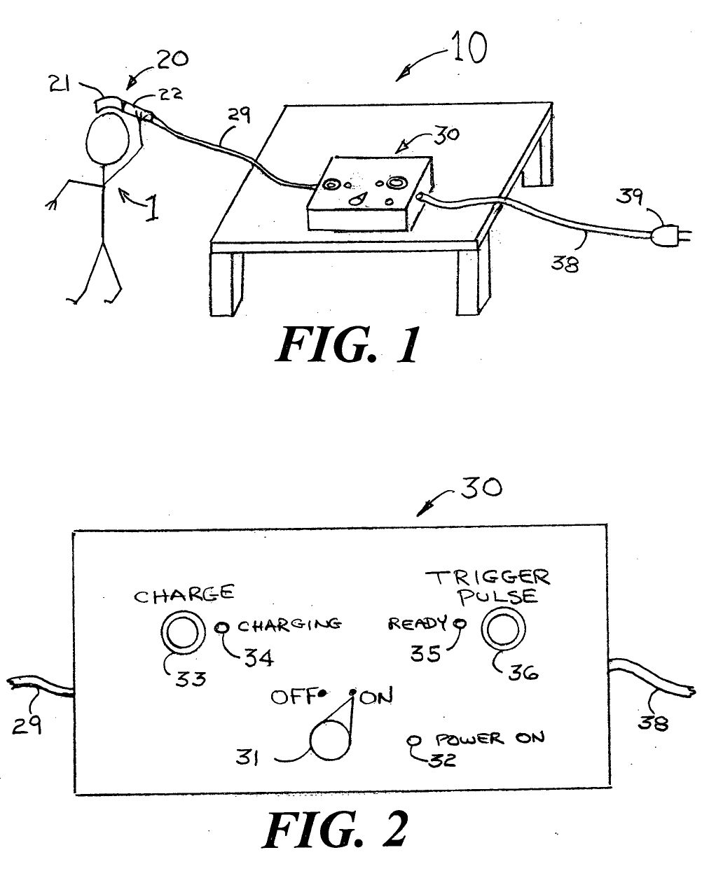

FIG. 1 illustrates the magnetic pulse system 10 that includes a head unit 20 and a table unit 30 that is connected to a source of electrical power by means of the electric power cable 38. The table unit 30 might have its power cable 38 with the plug 39 plugged into a-c line power or it could connect into another source of power such as the cigarette lighter in an automobile. It is also envisioned that the table unit 30 could be battery operated using either a rechargeable or a primary battery that could be either internally or externally located. The same table unit 30 could be designed to be connected at different parts of its circuitry to either the a-c line power receptacle, a car's cigarette lighter or a battery. The head unit 20 has a magnetic pulser portion 21 connected to a handle 22 with the magnetic pulser portion 21 being placed onto the head of a patient 1. The table unit 30 is connected to head unit 20 by means of an electrical connecting cable 29.

FIG. 2 illustrates the arrangement of the switches and indicator lights typically located on the top surface of the table unit 30. These switches and indicator lights (some or all of which could also be placed on the side of the table unit 30 or on the head unit 20) are used by the patient to operate the magnetic pulse system 10. Specifically, the ON-OFF switch 31 turns the system power on; the ON-state being indicated (for example) by a red POWER ON indicator light 32. It is readily conceived that there is no ON-OFF switch 31 with the table unit 30 being energized when it is plugged into a power source. A CHARGE push-button switch 33 when pushed and released would cause the capacitors 27 within the handle 22 of the head unit 20 to begin charging. The fact that the capacitors 27 are being charged would be indicated by an amber CHARGING indicator light 34. When the capacitors of the head unit 20 are fully charged, the READY indicator light 35 (typically green) would illuminate and the CHARGING indicator light 34 would be turned off. The patient would then grasp the head unit 20 by its handle 22 and place the magnetic pulse portion 21 of the head unit 20 at an appropriate position on his head. By pressing and releasing the TRIGGER PULSE push-button switch 36, the capacitors 27 in the head unit 20 would discharge a high intensity electric current through the coil windings 23 of the magnetic pulse portion 21 of the head unit 20 thus creating an intense magnetic pulse that is applied onto the neurons of the patient's brain. The READY indicator light would then be turned off. Although various possible colors for the indicator lights have been suggested herein, it is conceived that any combination of indicator lights as to number, color and location could be used with the table unit 30 or one or more switches or indicator lights could be located on the head unit 20. Also, any of these lights (particularly the CHARGING light 34) could be flashing to indicate a process that is going on. It is envisioned that the TRIGGER PULSE switch 36 might advantageously be placed on the head unit 20.

Once the capacitor discharge occurs, the CHARGE push-button can be pushed again and the patient can apply a second high intensity, short duration magnetic pulse to his head. Any number of additional pulses could be applied by the patient to his head or neck region if he believes that it would decrease the intensity or duration of his migraine headache. It is conceived that the table unit 30 would include a counter that counts the total number of pulses between visits to the patient's doctor so that the use of the magnetic pulse system 10 can be monitored.

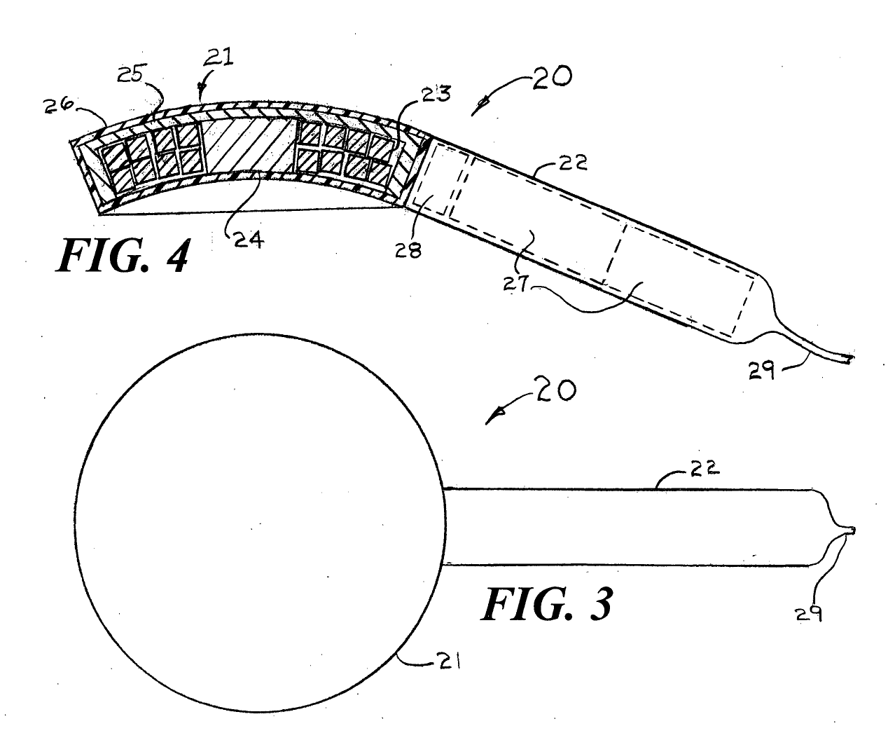

FIG. 3 is a top view of the head unit 20 showing the magnetic pulser portion 21 to which a handle 22 is attached. The connecting cable 29 joins the handle 22 to the table unit 30. The connecting cable 29 is used to provide a high voltage at a low electric current to charge the capacitors 27 that are located in the handle 22. For example, if creating the magnetic pulse requires 8,000 Amperes for 100 microseconds through its wire coil 23, and if the charge efficiency of the capacitors is 50%, then an electric current through the connecting cable 29 for 10 seconds would require only a comparatively low level of 0.16 Amperes of electric current. By this means, even a comparatively small gauge wire could be used to connect the table unit 30 to the head unit 20. A time between pulses of 10 seconds would be quite acceptable. A time as short as one second between pulses would increase the electric current in the connecting cable 29 to only 1.6 Amperes which still would allow a small gauge for the wires in the cable 29. Having a light weight connecting cable 29 is advantageous for easy use by the patient. This is accomplished by placing the capacitors 27 in the head unit 20 instead of the table unit 30.

FIG. 4 is a partial longitudinal cross section of the head unit 20. The head unit 20 consists of a magnetic pulser portion 21 (that is shown in cross section) and a handle 22. The handle 22 includes at least one capacitor 27 and control circuitry 28 each of which are shown as dotted lines within the handle 22 of the head unit 20. Although two capacitors 27 are shown in the handle 22, it is envisioned that any combination of one or more capacitors connected in series and/or in parallel can be used for providing the high intensity electric current pulse into the magnetic coil 23 so as to create the required high intensity, short duration magnetic pulse to treat the migraine headache.

The magnetic pulser portion 21 includes a conducting wire coil 23 through which an electrical current pulse is used to create the high intensity, short duration magnetic pulse that is required to abort a migraine headache. To create a magnetic pulser portion 21 with the lowest possible weight, the coil 23 is optimally made from insulated square or rectangular wire that is made from copper or aluminum. In point of fact, the lowest possible weight is obtained by the use of aluminum wire for the coil 23.

Also shown in FIG. 4 is a ferromagnetic center section 24 and a surrounding ferromagnetic section 25. The two ferromagnetic sections 24 and 25 can be used to enhance the intensity of the magnetic field pulse that is created by the magnetic pulser portion 21. To decrease eddy current loss in the ferromagnetic sections 24 and 25, those ferromagnetic components can be made in the form of laminations or they could be formed by powdered metal metallurgy or they could be formed using a molded ferrite. If a powdered metal ferromagnetic piece is used or if a ferrite is used, either of these could be formed or molded as a single piece with the coil 23 being placed inside that single piece of ferromagnetic material. It should also be understood that the magnetic pulser portion 21 could create a sufficiently intense magnetic pulse without the use of any ferromagnetic material; i.e., with just the wire coil 23.

As seen in FIGS. 1 and 4, a preferred shape for the magnetic pulser portion 21 is the sector of a sphere. The concave bottom surface of the magnetic pulser portion 21 allows a more intense magnetic field pulse to be applied to the brain as compared to a flat surface where the magnetic pulser portion 21 used the same electric current pulse. Although a flat magnetic pulser portion 21 is envisioned, (with or without ferromagnetic sections) a more optimum design is to have a magnetic pulser portion 21 whose concave surface is designed to meet with the normal curvature of a human head.

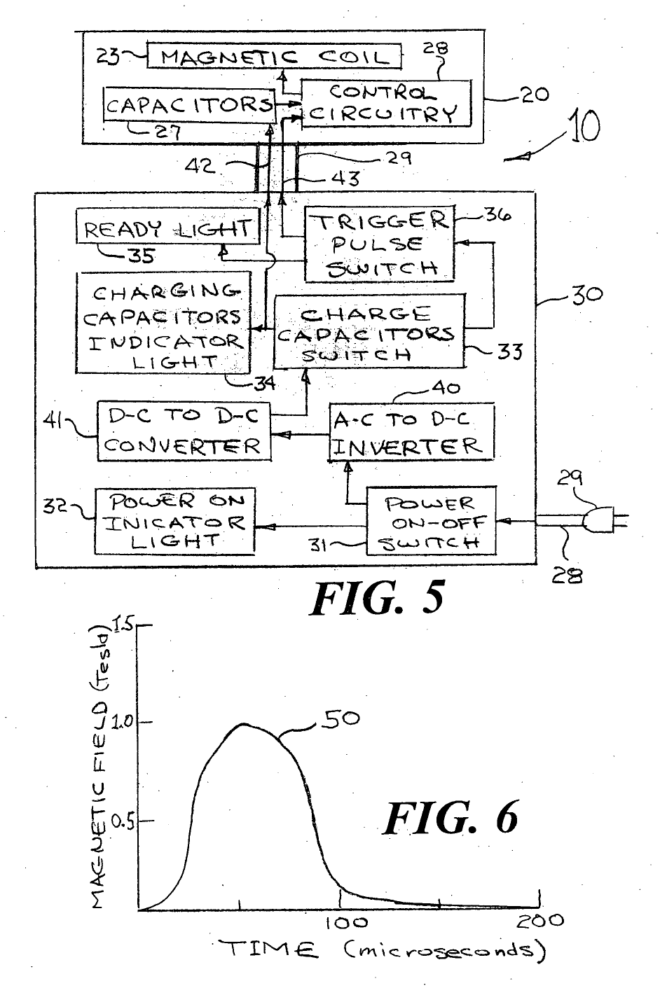

FIG. 5 is a simplified block diagram of the magnetic pulse system 10. In FIG. 5, the table unit 30 is connected to an outside source of electric power by means of the power cable 28 and the plug 29. The power enters the table unit 30 through a power ON-OFF switch 31. The power ON-state is indicated by the POWER ON indicator light 32. An a-c to d-c inverter 40 can be used to convert the a-c line voltage to a d-c voltage that is fed into the d-c to d-c converter 41. If the power source is from a car's cigarette lighter or from a battery, the a-c to d-c inverter 40 could be bypassed with the d-c power source being fed directly into the d-c to d-c converter 41. The output of the d-c to d-c converter 41 would typically be the high voltage (typically 500 to 1,000 volts d-c) that is required to charge the capacitors 27 of the head unit 20. When the charge capacitors switch 33 is pushed and released, the high voltage goes across the line 42 in the connecting cable 29 to the capacitors 27 of the head unit 20. While charging, the CHARGING capacitors indicator light 34 is illuminated or it may be flashing. When the capacitors 27 are fully charged, the READY indicator light 35 is illuminated and the patient can press the trigger pulse switch 36 to cause the capacitors 27 to discharge their stored charge into the magnetic coil 23 to create the required high intensity, short duration magnetic pulse to treat the migraine headache. A line 43 in the connecting cable 29 can be used to activate the control circuitry 28 in the head unit 20 to cause the capacitors 27 to discharge through the magnetic coil 23. It is clearly envisioned that there are alternative ways to create the required high intensity, short duration magnetic pulse. However, the feature of placing the capacitors 27 in the head unit 20 which allows a very light weight and flexible connecting cable 29 to provide the charging current for those capacitors 27 is certainly a novel feature of this invention. Placing the capacitors 27 in the handle 22 of the head unit 20 is also a novel feature that optimizes the system design. Still further, making the magnetic coil 23 (with or without ferromagnetic structures) out of square or rectangular aluminum wire is a novel design feature of the head unit 20 which improves the portability of the magnetic pulse system 10 by reducing the weight of the head unit 20.

FIG. 6 is a graph 50 of the intensity of the magnetic pulse as a function of time with time in microseconds being the abscissa and the ordinate being the magnetic field strength in Tesla. The curve of the graph 50 of FIG. 6 would be the magnetic field strength taken along the centerline of the magnetic pulse portion 21 at a distance of 1.0 cm below its bottom, concave surface. The time tₘ in FIG. 6 is the time at which the pulse reaches its maximum intensity. The optimum values for tₘ lie between 10 and 1,000 microseconds. From actual experience with treating aura in human patients, the more optimum value for tₘ is between 20 and 200 microseconds.

It should be understood that in order to be useful to a migraine patient, the magnetic pulse system 10 must have several distinct characteristics that are different from currently available systems for TMS or repetitive Transcranial Magnetic Stimulation (rTMS). Specifically, the inventive concept of the present invention includes the fact that the magnetic pulse system 10 is readily portable, has preset operating parameters that are not adjustable by the patient, can be placed on the patient's head by the patient and is turned on and off by the patient. “Readily portable” can be defined as having a weight for the entire magnetic pulse system 10 of less than 10 kg with the head unit having a weight of less than 3 kg. More optimally, the entire system should weigh less than 5 kg and the head unit less than 1.5 kg. One of the well known types of TMS and rTMS equipment is the Cadwell MES-10 unit that is operated by a physician and not by a patient, has operational parameters that are adjustable by the physician as it is being used (i.e., the parameters are not preset), has a magnetic coil that is placed on a patient by an attending physician, and since the entire system weighs 34 kg it is certainly not readily portable so as to be with the patient wherever he might need it. Furthermore, the capacitors of the Cadwell unit are not located in its head unit so that the cable connecting its table unit to its head unit is extremely heavy, stiff and has a large diameter. To be useful for its intended purpose, the magnetic pulse system 10 should have operating parameters that are preset by an attending physician or preset at the factory. These operating parameters can include one or more of the following attributes: the peak intensity of the magnetic field at a distance of 1.0 cm beneath the head unit, the time period of each magnetic pulse; the maximum allowed repetition rate of the magnetic pulses; and the total number of pulses to be delivered when the magnetic pulse system 10 is turned on. Once these parameters are set, the patient would operate the system 10 by placing it on his head and then turning the system on and then off after the treatment for the migraine headache has been terminated. It may be desirable for the patient to turn the system on but a timer could automatically turn the system off after a preset period of time.

Since the aura of a migraine headache might occur at any time, and since the patient may have only 20 minutes to use the magnetic pulse system 10, each patient would want to have a system in relatively close proximity. For example, the patient would want to have the system at home, and/or at work, and/or in his car. The magnetic pulse system 10 would optimally be sufficiently portable to be taken with the patient on a vacation or on a business trip.

It is also envisioned that the magnetic pulse system 10 could include a memory for recording various parameters of the magnetic pulse system including the setting of the magnetic field intensity or how many pulses have been used by the patient. Within a limited range, it is envisioned that the patient could set different levels for the magnetic field intensity in order to determine that level that is most effective in preventing a migraine headache. It is further envisioned that the magnetic pulse system 10 as described herein could be used for the treatment of other disorders such as depression, pain, epilepsy (especially acute and/or febrile seizures), bi-polar disease and other disorders of the nerves or brain such as trigeminal neuralgia and tinnitus. It is also envisioned that the system could be adapted for measuring nerve conduction velocity in various parts of the human body.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

What is claimed is:

1. A magnetic pulse system for the treatment of migraine headaches, the system including:
a head unit designed for placement onto the head of a migraine patient, the head unit having an electromagnetic coil and at least one capacitor, the combination being capable of acting together to provide a high intensity, short duration magnetic pulse onto some portion of the patient's brain, the peak field at the surface of the brain being at least 0.1 Tesla;
a table unit that receives electric power from an outside power source or from an internal battery, the table unit providing an electric current to the head unit via a connecting cable, the electric current being used to charge the at least one capacitor in the head unit; and,
the magnetic pulse system also having a patient operated switch to cause the charged at least one capacitor in the head unit to discharge through the electromagnetic coil to create the high intensity, short duration magnetic pulse onto the brain of the migraine patient.

2. The magnetic pulse system of claim 1 also including an electrical switch operated by the migraine patient to initiate charging of the at least one capacitor in the head unit.

3. The magnetic pulse system of claim 2 also including an indicator light that indicates when the at least one capacitor is being charged.

4. The magnetic pulse system of claim 2 also including an indicator light that indicates when the at least one capacitor is fully charged.

5. The magnetic pulse system of claim 1 where the head unit utilizes one or more ferromagnetic sections located in close proximity to the electromagnetic coil to decrease the amount of electric current required to provide a specific level of magnetic pulse intensity.

6. The magnetic pulse system of claim 5 where the ferromagnetic material is molded from a powdered ferromagnetic metal.

7. The magnetic pulse system of claim 5 where the ferromagnetic material is formed from a ferrite material.

8. The magnetic pulse system of claim 5 where the ferromagnetic material is formed from laminations of a ferromagnetic metal.

9. The magnetic pulse system of claim 1 where the head unit and the table unit cooperate to deliver at least one high intensity, short duration magnetic pulse each time a switch is triggered by the migraine patient to deliver the charge from the at least one capacitor into the electromagnetic coil.

10. The magnetic pulse system of claim 1 where the time to reach the peak magnitude of the magnetic pulse is between approximately 10 microseconds and approximately 1,000 microseconds.

11. The magnetic pulse system of claim 1 where the system includes at least one operating parameter that is preset by a physician.

12. The magnetic pulse system of claim 1 where the head unit of the magnetic pulse system has a magnetic pulser portion that has the shape of a spherical sector so as to maximize the intensity of the magnetic pulse onto the neurons of the brain for a given level of electric current pulse in the magnetic coil of the magnetic pulser portion, the concave surface of the spherical sector having approximately the same curvature as a human head.

13. The magnetic pulse system of claim 1 where the head unit of the magnetic pulse system has a magnetic pulser portion and a handle attached to the magnetic pulser portion.

14. The magnetic pulse system of claim 13 where the at least one capacitor is placed inside of the handle of the head unit.

15. The magnetic pulse system of claim 13 where the magnetic pulser portion of the head unit has an electromagnetic coil that is made from square or rectangular wire.

16. The magnetic pulse system of claim 15 where the metal of the square or rectangular wire of the electromagnetic coil is aluminum.

17. The magnetic pulse system of claim 1 where the weight of the entire magnetic pulse system is less than 10 kg so that the system is readily portable.

18. The magnetic pulse system of claim 1 where the combined weight of the head unit and its connecting cable to the table unit is less than 1.5 kg so that the head unit can be readily placed onto the patient's head by the patient.

19. A method for the treatment of a migraine headache using a magnetic pulse system, the method including the following steps:
(a) sensing of an aura of a migraine headache by a migraine patient;
(b) charging at least one capacitor in a head unit of a magnetic pulse system from a table unit that has an outside power source or an internal battery, the head unit and the table unit being electrically connected by means of a connecting cable;
(c) placing the head unit onto the head of the migraine patient by the patient himself;
(d) having the migraine patient trigger a switch to cause the head unit to create a high intensity, short duration magnetic pulse onto the patient's brain in the vicinity of the brain where the aura is occurring.

20. The method of claim 19 further including the step of applying at least one magnetic pulse having a time duration between approximately 10 microseconds and approximately 1,000 microseconds.

21. The method of claim 19 further including the step of turning an ON-OFF switch by the patient first to the ON position and then to the OFF position after the magnetic pulse has been applied to the patient's head.

22. The method of claim 19 further including the step of having the patient turn the ON-OFF switch to the ON position and then having the magnetic depolarizer system automatically turn the system off after a preset time period.

23. The method of claim 19 further including the step of having a physician set at least one operating parameter of the system.

24. The method of claim 19 further including the step of having the patient place the head unit with the magnetic pulser portion placed onto his head in close proximity to the patient's occipital lobe of his brain.

25. A method for treating a migraine headache in a patient with a readily portable magnetic pulse system, the method including the following steps:
a) detecting the onset of a migraine headache;
b) charging a capacitor in a head unit by means of an electric current from a connecting cable attached to a table unit of a magnetic pulse system; and
c) generating a high intensity, short duration magnetic pulse having a peak intensity at some portion of the patient's brain of at least 0.1 Tesla.

26. The method of claim 25, wherein the step of detecting the onset of the migraine headache is accomplished by the patient.

27. The method of claim 25 further including the step of applying at least one magnetic pulse having a time duration between approximately 10 microseconds and approximately 1,000 microseconds.

28. The method of claim 25 further including the step of turning an ON-OFF switch by the patient first to the ON position and then to the OFF position after the magnetic pulse has been applied to the patient's head.

29. The method of claim 25 further including the step of having the patient turn the ON-OFF switch to the ON position and then having the magnetic depolarizer system automatically turn the system off after a preset time period.

30. The method of claim 25 further including the step of having a physician set at least one operating parameter of the system.

31. The method of claim 25 further including the step of having the patient place the head unit with the magnetic pulser portion placed onto his head in close proximity to the place where the patient has pain from his migraine headache.
